(19) United States
(12) Patent Application Publication
(10) Pub. No.: US 2025/0135386 A1
(43) Pub. Date: May 1, 2025

(54) PROCESS FOR ERADICATING/REDUCING CORONAVIRUS AND OTHER SMALL PATHOGENS IN THE INDOORS AIR

(71) Applicant: Kailash C. Vasudeva, Kitchener (CA)

(72) Inventor: Kailash C. Vasudeva, Kitchener (CA)

(21) Appl. No.: 18/683,499

(22) PCT Filed: Aug. 12, 2022
(86) PCT No.: PCT/CA2022/051233

§ 371 Date: Feb. 13, 2024

Related U.S. Application Data
(60) Provisional application No. 63/298,598, filed on Jan. 11, 2022.
(60) Provisional application No. 17/402,489, filed on Aug. 13, 2021.

(30) Foreign Application Priority Data
Aug. 13, 2021 (CA) 3128201

(51) Int. Cl. B01D 46/00; A61L 9/00; A61L 101/26; B01D 39/08; B01D 39/16; F24F 8/108; F24F 8/24; F24F 13/08

(52) U.S. Cl. CPC B01D 46/0028; A61L 9/00; B01D 39/083; B01D 39/1615; B01D 46/0045; F24F 8/108; F24F 8/24; F24F 13/08; A61L 2101/26; A61L 2209/14; A61L 2209/16; B01D 2239/0258; B01D 2239/0407; B01D 2239/0442; B01D 2239/0478; B01D 2239/0613; B01D 2239/0618; B01D 2273/30; B01D 2279/50; B01D 2279/65

(57) ABSTRACT

Standalone units and modules for providing purified air are described herein. The standalone units and the modules include antimicrobial protection as provided by one or more one antimicrobial elements being configured to receive the air from a filter and eradicate or remove small pathogens from the air to provide purified air. Methods of providing purified air are also described herein.

### CROSS-REFERENCE

The present application claims the benefit of Canadian Patent Application Number 3,128,201 entitled “Process for Eradicating/Reducing Coronavirus and Other Small Pathogens in the Indoors Air” filed Aug. 13, 2021; U.S. patent application Ser. No. 17/402,489 entitled “Process for Eradicating/Reducing Coronavirus and Other Small Pathogens in the Indoors Air” filed Aug. 13, 2021, and U.S. Provisional Patent App. No. 63/298,598 “Multiple Stage Process for Eradicating/Reducing Small Pathogens in the Indoors Air” filed Jan. 11, 2022, the entire contents of each of which are hereby incorporated by reference herein.

### TECHNICAL FIELD

The embodiments disclosed herein relate to air purifiers and/or sanitizers, and more specifically, to systems, devices and methods for eradicating and/or reducing Coronavirus and other small pathogens from indoor air.

### BACKGROUND

As the pandemic of the coronavirus has spread around the world, it has been established that the coronavirus is capable of being airborne. This means that individuals in indoor environments with heating, ventilation and air conditioning (HVAC) systems, or other air circulation systems, presently installed are at a risk of infection of the coronavirus. These indoor environments include schools, colleges, universities, workplaces such as but not limited to factories, offices, shops and shopping malls, hospitals, old age care centers, intensive care centers, airplanes, train coaches, public buses, taxies and the like.

Typical air circulation systems where either large crowds are present, or in laboratory settings, or critical care centers, use high-efficiency particulate absorbing (HEPA) filters, particularly of type H13-14 with a Minimum Efficiency Reporting Value (MERV) rating in a range of about 13 to 20, that have up to 99.99 percent filtration efficacy.

Typical air circulation systems for commercial applications use ePM1 filters having a MERV rating in a range of about 9 to 13, with a filtration efficacy of more than 80 percent.

Typical HEPA filter systems can contain particulates from >5 microns to <5-microns size.

Coronavirus, or its variants, or other small pathogens are very small, such as but not limited to being about 0.06 to about 0.14 microns. Therefore, these small pathogens cannot be filtered by the filters note above.

It is extremely difficult to improve upon existing HVAC systems installed in commercial, residential and health services set-ups.

Therefore, there is a need for new systems, devices and methods for eradicating and/or reducing coronavirus and/or other small pathogens from indoor air.

### SUMMARY

In accordance with a broad aspect, a standalone unit for providing purified air is described herein. The standalone unit includes a housing including an air inlet to receive the air and an air outlet to provide purified air; a fan positioned within the housing and between the air inlet and the air outlet, the fan configured to control a rate of airflow through the standalone unit; a filter positioned within the housing and configured for removing particulates from the air received from the air inlet to provide filtered air; and at least one antimicrobial element positioned within the housing downstream from the filter to receive the filtered air from the filter, the at least one antimicrobial element being configured to receive the filtered air from the filter and eradicate or remove small pathogens from the filtered air to provide the purified air.

In at least one embodiment, the antimicrobial element includes a filter having a filter material and an antimicrobial coating at least partially applied to the filter material.

In at least one embodiment, the filter material is entirely coated with the antimicrobial coating.

In at least one embodiment, the antimicrobial coating comprises one or more salts from an ionic liquid being applied to the filter material.

In at least one embodiment, the one or more salt of the antimicrobial coating includes cations and anions crystallized on the filter material.

In at least one embodiment, the cations include one or more of imidazolium, pyridnium, piperidinium, pyrrolidinium, quinolinium, morpholinium, quaternary phosphonium and quaternary ammonium cations.

In at least one embodiment, the anions includes one or more of tetrafluoroborate, hexafluorophosphate, methylsulfate, octylsulfate, acesulfame, halide ions, bis(trifluoromethyl)sulfonylamide, bis(trifluoromethyl) amide, dicyanamide, and trifluoromethylsulfonate.

In at least one embodiment, the antimicrobial coating includes one or more metal nanoclusters.

In at least one embodiment, the metal nanoclusters include copper nanoclusters.

In at least one embodiment, the antimicrobial element includes a copper mesh positioned within the housing downstream from the filter.

In at least one embodiment, the antimicrobial element includes a set of vortex tubes.

In at least one embodiment, the set of vortex tubes each have an inner surface at least partially coated with an antimicrobial material.

In at least one embodiment, the set of vortex tubes each have an inner surface entirely coated with the antimicrobial material.

In at least one embodiment, the antimicrobial material a copper-based material.

In at least one embodiment, the antimicrobial material is copper.

In at least one embodiment, the set of vortex tubes include left-hand and right-hand spiraling vortex tubes.

In at least one embodiment, the standalone unit further comprises a third filter positioned downstream from the set of vortex tubes and adjacent to the outlet, the third filter having a copper-based material.

In accordance with another broad aspect, a module for a heating, ventilation and air conditioning (HVAC) system for purifying air of the HVAC system is described herein. The module is configured to be positioned within a portion of the HVAC system to receive the air of the HVAC system. The module includes an antimicrobial element configured to eradicate or remove small pathogens from the air to provide purified air.

In at least one embodiment, the module is configured to be positioned within a duct of the HVAC system.

In at least one embodiment, the module is configured to be positioned within a ceiling air deflector of the HVAC system.

In at least one embodiment, the systems, devices and methods for eradicating and/or reducing coronavirus and/or other small pathogens from indoor air include multiple stages of control. In at least one embodiment, this may include a first stage of control, a second stage of control, a third stage of control and/or additional stages of control.

In at least one embodiment, the first stage of control is a filter for the air circulation system including portable room air purifier systems using the recommended HEPA filters type H13-14 which has the MERV rating of 13-20 to have a filtration efficacy up to 99.99 percent.

In at least one embodiment, the second stage of control is for the particles and bio-organisms of sub-micron size, includes coronavirus for SARS, MERS and COVID-19 which would not be filtered by the best HEPA filter.

In at least one embodiment, the air circulation system includes a HEPA filter type H13-14 with MERV rating of 13-20.

In at least one embodiment, the HEPA filter is replaceable.

In at least one embodiment, the function of the HEPA filter can be enhanced by spraying salt solution until dried and crystallized. As an alternative an additional replaceable filter with salt spray could be added next to the HEPA filter as a second stage of protection.

In at least one embodiment, the function of the HEPA filter can be enhanced by treating it with ionized liquid embedded with copper nanoclusters. As an alternative an additional replaceable filter with ionized liquid embedded with copper nanoclusters could be added next to the HEPA filter as a second stage of protection.

In at least one embodiment, the air circulation system includes a multiple layers of copper mesh installed as extended antimicrobial surface next to HEPA filter. This could function as a second stage of protection.

In at least one embodiment the second stage of protection could be our proprietary vortex system with spiraling antimicrobial surfaces that help in sanitizing and structuring the air.

In at least one embodiment an additional stage of protection could be a cluster of UVC lights.

In at least one embodiment, the UVC light has the wavelength of 265 nm.

In at least one embodiment, the UVC light lamps are installed perpendicular to the airflow.

In at least one embodiment, the air circulation system includes an efficacy monitoring system, such as but not limited to a digital anemometer that shows air flow rate or air exchange rate.

In at least one embodiment, the air circulation system includes sampling probes that could be installed before and after the second/third stage of protection as per ANSI/ASHRAE procedure.

In at least one embodiment, the monitoring system can be integrated into a sensor/alarm system forewarning the presence of harmful pathogens.

In at least one embodiment, the air circulation system the air cleaning efficiency can be enhanced by increasing the per hour air exchange rate.

These and other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a cross-sectional view of a standalone room air purifying unit that uses a high efficiency HEPA filter as a first stage of protection and a coated/treated filter as a second stage of protection, according to at least one embodiment described herein.

FIG. 2 is a cross-sectional view of a standalone room air purifying unit that uses a high-efficiency particulate absorbing (HEPA) filter as a first stage of protection followed by multiple layers of an antimicrobial surface (e.g., a copper mesh) as a second stage of protection, according to at least one embodiment described herein.

FIG. 3 is a cross-sectional view of a standalone room air purifying unit that uses a HEPA filter as first stage of protection and a set of vortex tubes with spiraling antimicrobial surfaces as a second stage of protection, according to at least one embodiment described herein.

FIG. 4a is a perspective view of a standalone room air purifying unit having a preconfigured cluster of ultraviolet C (UVC) lights positioned before an exit grill as a third stage of protection, according to at least one embodiment described herein.

FIG. 4b is a cross-sectional view of the standalone room air purifying unit of FIG. 4B along line 4b-4b.

FIG. 5 is a housing of a heating, ventilation and air conditioning (HVAC) unit with an inlet duct and filter slots, according to at least one embodiment described herein.

FIG. 6 is a cutaway view of the HVAC unit of FIG. 5 having a cooling unit and an element for a second stage of protection.

FIG. 7 is a cross-sectional view of a module having extended antimicrobial surfaces retrofitted in an existing HVAC duct, according to at least one embodiment described herein.

FIG. 8 is a cross-sectional view of a module of vortex tubes retrofitted into an existing HVAC duct, according to at least one embodiment described herein.

FIG. 9a is a perspective view of a ceiling air deflector outlet of an HVAC system, according to at least one embodiment described herein.

FIG. 9b is a cross-sectional view of the ceiling air deflector outlet of FIG. 9a.

FIG. 9c is a magnified portion of the cross-sectional view of the ceiling air deflector outlet of FIG. 9a.

FIG. 10a is a front perspective view of a standalone room air purifying unit with multi-stage protection, according to at least one embodiment described herein. FIG. 10b is a rear perspective view of the standalone room air purifying unit

of FIG. 10a.

FIG. 10c is a cross-sectional view of the standalone room air purifying unit of FIG. 10a.

FIG. 10d is a front view of a set of vortex tubes of the standalone room air purifying unit of FIG. 10a.

Further aspects and features of the example embodiments described herein will appear from the following description taken together with the accompanying drawings.

### DETAILED DESCRIPTION

Various systems, devices and methods will be described below to provide an example of one or more embodiments. No embodiment described below limits any claimed embodiment and any claimed embodiment may cover systems, devices or methods that differ from those described below. The claimed embodiments are not limited to systems, devices and methods having all of the features of any one system, device or method described below or to features common to multiple or all of the systems, devices and methods described below. It is possible that a system, device or method described below is not an embodiment of any claimed embodiment. Any embodiment disclosed below that is not claimed in this document may be the subject matter of another protective instrument, for example, a continuing patent application, and the applicants, inventors or owners do not intend to abandon, disclaim or dedicate to the public any such embodiment by its disclosure in this document.

In general, the present document is directed to systems, devices and methods for purifying air.

In at least some embodiments described herein, the systems, devices and methods described for purifying air are suitable for use in indoor environments. Typically, similar systems, devices and methods currently in use are insufficient for removing small pathogens, including but not limited to Coronavirus for severe acute respiratory syndrome (SARS), Middle East respiratory syndrome (MERS) and COVID-19.

The problems to be solved by the present disclosure are not limited to the above-mentioned problem(s), and other problem(s) not mentioned can be clearly understood by those skilled in the art from the following description.

Herein, the term antimicrobial refers to a substance that destroys or inhibits the growth of microorganisms, and especially pathogenic microorganisms.

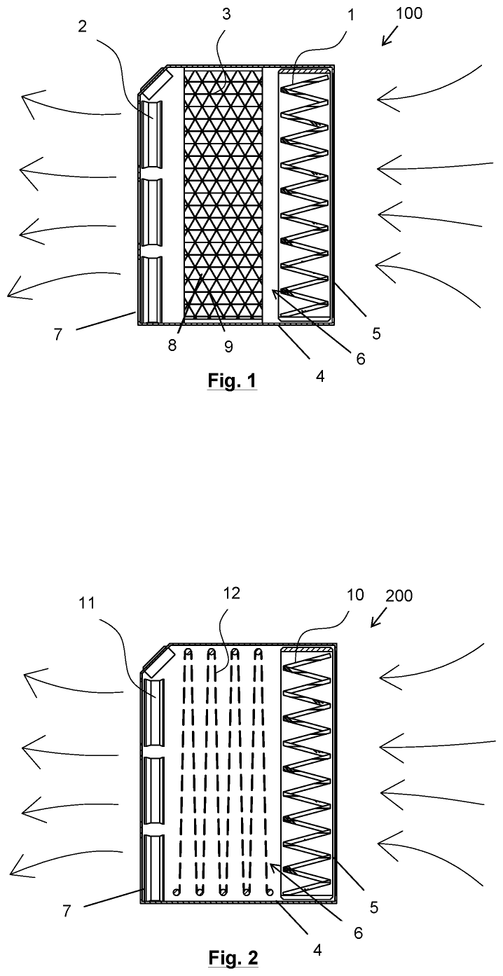

FIG. 1 shows a cross-sectional view of a standalone room air purifying unit 100 according to at least one embodiment described herein. Standalone room air purifying unit 100 includes a HEPA filter 1 as first stage of protection and a second (e.g., coated/treated) filter 3 as second stage of protection. Each of HEPA filter 1 and second filter 3 are held within a housing 4. Second filter 3 is positioned downstream of the HEPA filter 1 within housing 4. Standalone room air purifying unit 100 also includes an inlet 5. Air is drawn into a chamber 6 defined by housing 4 through inlet 5 by one or more fans 2. Fan(s) 2 are positioned next to an outlet 7. Air is drawn into chamber 6 through inlet 5, subsequently through HEPA filter 1 and second filter 3, and then emitted from chamber 6 through outlet 7 by fan(s) 2.

Second filter 3 includes a filter material (i.e., a woven or felted fabric made from wool, cotton, or a similar fiber) 8 and an antimicrobial coating 9 at least partially applied to the filter material 8, for example, is sprayed onto the filter material.

In at least one embodiment, the filter material 8 is entirely coated with antimicrobial coating 9.

In at least one embodiment, the antimicrobial coating 9 is formed from a salt (e.g., an ionic liquid), optionally having metal nanoclusters mixed therein.

In at least one embodiment, the ionic liquid includes one or more ionic components. In at least one embodiment, the one or more ionic components includes a cationic component that, for example, triggers electrostatic interactions impacts binding mechanisms of one or more small pathogens.

In at least one embodiment, the one or more ionic components includes a one or more cation components and one or more anion components. In at least one embodiment, the one or more cation components includes one or more of imidazolium, pyridnium, piperidinium, pyrrolidinium, quinolinium, morpholinium, quaternary phosphonium and quaternary ammonium. In at least one embodiment, the one or more anion components includes one or more of tetrafluoroborate, hexafluorophosphate, methylsulfate, octylsulfate, acesulfame, halide ions (e.g., chlorine ions, bromine ions and/or iodine ions), bis(trifluoromethyl)sulfonylamide, bis(trifluoromethyl) amide, dicyanamide, and trifluoromethylsulfonate.

In at least one embodiment, the one or more cation component of the ionic liquid is configured to disrupt a membrane of the small pathogens upon contact, interfere with DNA therein and/or expose intracellular material to lead to the eradication of the small pathogens from the air.

In at least one embodiment, the ionic liquid includes one or more metal nanoparticles, such as but not limited to copper nanoclusters.

In at least one embodiment, the ionic liquid includes one or more metal nanoparticles, such as but not limited to nanoparticles containing one or more of titanium, silver and zinc. In some embodiments, the metal nanoparticles being positioned on the second filter 3 generate free radicals and lead to induction of oxidative stress (i.e., reactive oxygen species; ROS). The generated ROS can damage and destroy the cellular components of the pathogens irreversibly, (e.g., membrane, DNA, protein and mitochondria), resulting in cell death.

In at least one embodiment, the ionic liquid can be mixed with water and applied (e.g., sprayed) to the filter material 8. As the water evaporates from the surface of the filter material 8, the ionic liquid components remain adhered to the filter material 8 to form antimicrobial coating 9.

In at least one embodiment, the first stage of protection (e.g., HEPA filter 1) may include type H13-14 HEPA filters, or the like, which have a MERV rating of in a range of about 13 to 20 to have a filtration efficacy up to about 99.99 percent.

In at least one embodiment, the first stage of control (e.g., HEPA filter 1) may be a modified HEPA filter. For example, in at least one embodiment, the function of the HEPA filter may be enhanced by spraying a salt solution onto the HEPA filter and allowing the salt solution to dry and crystallize.

As an alternative, in at least one embodiment, an additional replaceable filter having a salt solution applied thereto could be added next to the HEPA filter 1 as a second stage of protection.

In at least one embodiment, the second stage of protection provides for eradicating and/or reducing a presence of one or more small pathogens in indoor air. Herein, the term “small pathogen” is intended to refer to any microbe that can cause damage in a host. The one or more small pathogens may therefore include, but are not limited to, bio-organisms of sub-micron size, including but not limited to Coronavirus for severe acute respiratory syndrome (SARS), Middle East respiratory syndrome (MERS) and COVID-19. As should be understood, these small pathogens would not be filtered by traditional HEPA filters.

In at least one embodiment, the function of the HEPA filter may be enhanced by treating it with an ionized liquid as previously described, optionally embedded with copper nanoclusters.

As an alternative, in at least one embodiment, an additional replaceable filter having an ionized liquid embedded with copper nanoclusters could be added next to the HEPA filter as a second stage of protection.

FIG. 2 shows a cross-sectional view of a standalone room air purifying unit 200 according to at least one embodiment described herein. Standalone room air purifying unit 200 uses a HEPA filter 10 as first stage of protection followed by multiple layers of an antimicrobial element 12 as an antimicrobial element as second stage of protection.

Each of the HEPA filter 10 and multiple layers of antimicrobial element 12 are held within a housing 4 of standalone room air purifying unit 200. The antimicrobial element 12 (or layers thereof) is positioned downstream of the HEPA filter 10 within housing 4. Standalone room air purifying unit 200 also includes an inlet 5. Air is drawn into a chamber 6 defined by housing 4 through inlet 5 by one or more fans 11. Fan(s) 11 are positioned next to an outlet 7. Air is drawn into chamber 6 through inlet 5, subsequently through HEPA filter 1 and coated/treated filter 3, and then emitted from chamber 6 through outlet 7 by fan(s) 2.

In at least one embodiment, the antimicrobial element 12 is a copper mesh. In at least one embodiment, the antimicrobial element 12 is made of a copper alloy. Copper and its alloys (e.g., brasses, bronzes, cupronickel, copper-nickel-zinc, and others) are natural antimicrobial materials.

In at least one embodiment, the antimicrobial element 12 is a single layer of a copper mesh formed from a copper wire. In at least one embodiment, the copper wire forming the mesh 12 has a diameter in a range of about 0.3 mm to about 1 mm. In other embodiments, the copper wire forming the mesh 12 has a diameter less than about 0.3 mm. In other embodiments, the copper wire forming the mesh 12 has a diameter greater than about 1 mm. In at least one embodiment, the antimicrobial element 12 is a copper mesh having an average pore size in a range of about 0.3 mm to about 1 mm. In other embodiments, the antimicrobial element 12 is a copper mesh having an average pore size less than about 0.3 mm. In other embodiments, the antimicrobial element 12 is a copper mesh having an average pore size greater than about 1 mm.

In at least one embodiment, the antimicrobial element 12 is a plurality of layers of a copper mesh, each layer of copper mesh being formed by a copper wire as described above. For example, the antimicrobial element 12 may include 1, 2, 3, 4, 5, 6, 7, 8, 9, 10 or more layers of copper mesh. In at least one embodiment, each layer of copper mesh may be spaced apart from a previous layer of copper mesh. In at least one embodiment, each layer of copper mesh may be positioned adjacent to and/or abutting at least one other layer of copper mesh. In at least one embodiment, the layers of the copper mesh may be arranged so that the wires of a first layer cover pores of an adjacent layer in such a way that there is negligible probability of pathogens passing through the copper mesh without contacting the copper mesh.

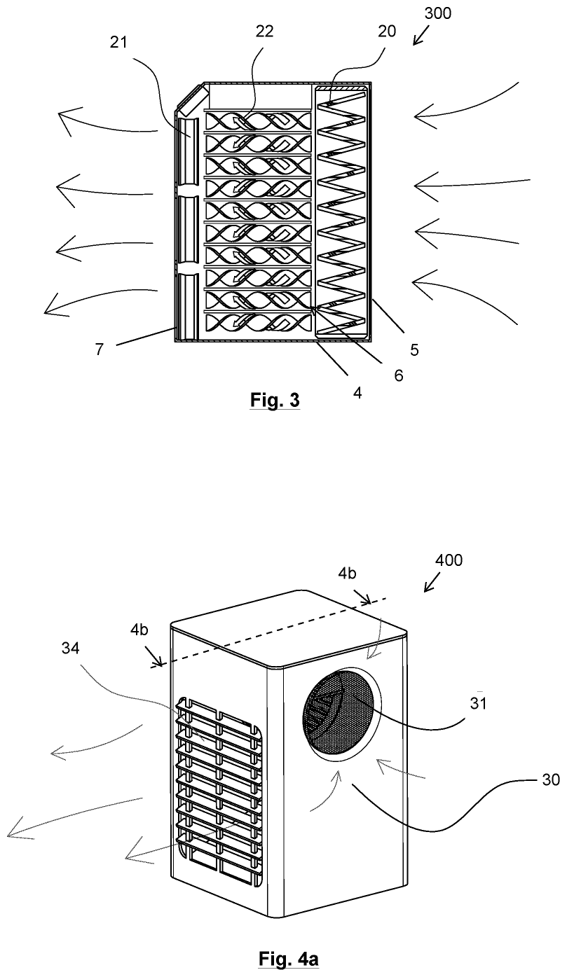

FIG. 3 shows a cross-sectional view of a standalone room air purifying unit 300 that uses a HEPA filter 20 as a first stage of protection and a set of vortex tubes 22 as an antimicrobial element, optionally including one or more coating materials thereon (e.g., on an inner surface thereof) that provides an antimicrobial effect, as a second stage of protection. In this embodiment, the air received from the HEPA filter 20 passes through the inside of the set of vortex tubes 22 and contacts an inner wall of the vortex tubes 22. The vortex tubes 22 provide for an increased contact time and an increased contact area between the filtered air and, in at least some embodiments, an antimicrobial coating material coated on an inner surface of the vortex tubes, relative to other elements described herein.

In at least one embodiment, the vortex tubes 22 include an antimicrobial material, such as but not limited to copper, or any other antimicrobial material, disposed on an inner surface for contact with the air therein. Each of the vortex tubes 22 has a diameter in a range of about 1 inch to about 6 inches, or in a range of about 1.5 inches to about 3 inches, or in a range of about 2 inches to about 3 inches, or of about 2 inches, or of about 3 inches. In at least one embodiment, the antimicrobial agent (e.g., copper) is present on a portion of an inner wall of each of the vortex tubes 22. In at least one embodiment, the antimicrobial agent (e.g., copper) is present on an entire inner wall of each of the vortex tubes 22.

The vortex tubes 22 are each configured to force air passing therethrough to have a vortex spinning action. For instance, each of the vortex tubes 22 may include a vortex generator (e.g., a nozzle) to induce the air passing therethrough to have a vortex spinning action. When the air having the vortex spinning action contains one or more pathogens, the pathogens contact the antimicrobial material coating the inner surfaces of the vortex tubes 22. In at least one embodiment, the vortex tubes 22 include both left hand and right hand spiral vortex tubes 22.

Each of HEPA filter 20 and vortex tubes 22 are held within a housing 4 of standalone room air purifying unit 300. The set of vortex tubes 22 are positioned downstream of the HEPA filter 20 within housing 4. Standalone room air purifying unit 300 also includes an inlet 5. Air is drawn into a chamber 6 defined by housing 4 through inlet 5 by one or more fans 21. Fan(s) 21 are positioned next to an outlet 7. Air is drawn into chamber 6 through inlet 5, subsequently through HEPA filter 20 and through one of the set of vortex tubes 22. The HEPA filter and the vortex tubes 22 are arranged to ensure that air must pass therethrough to reach outlet 7. Air is emitted from chamber 6 through outlet 7 by fan(s) 21.

FIG. 4a shows a perspective view of a standalone room air purifying unit 400 having a preconfigured cluster of ultraviolet C (UVC) lights 35 (see FIG. 4b) positioned before an exit grill 34 as a third stage of protection.

FIG. 4a shows standalone room air purifying unit 400 including a HEPA filter (not shown) as a first stage of protection and at least one of the elements according to at least one of the embodiments described above as providing a second stage or protection (e.g., second filter 3, antimicrobial element 12, set of vortex tubes 22, or some combination thereof). Each of the HEPA filter and the element providing the second stage of protection are held within a housing 30. For example, as shown in FIG. 4b, the element providing the second stage or protection is several layers of copper mesh 12 positioned downstream of the HEPA filter 1 within housing 4. UVC lights 35 are then positioned downstream from the element providing the second stage or protection

Unit 400 also includes drum-type centrifugal fan 33 that draws air from inlet 31 (see FIG. 4a) and pushes the air through a spiral path 36 within the housing 6. In this embodiment, layers of copper mesh 12 are positioned within spiral path 36. Air exits chamber 6 through outlet 34 after passing by (e.g., through) the UVC lights 35. In at least one embodiment, the UVC lights 35 are arranged to be perpendicular to a direction of flow of the air through outlet 34.

In at least one embodiment, the UVC lights 35 may provide a dosage of UVC light effective for killing about 90 percent of most of the pathogens, such as but not limited to a dosage in a range of about 2000 to about 8000 mJ/cm² with the wavelength of 265 nm as the UVC light destroys the DNA and cell structure of the virus.

In some embodiments, the forms of protection described above may be integrated into other forms to provide for eradicating and/or reducing a presence of one or more small pathogens in the air.

For example, without limiting the foregoing, in at least one embodiment, the elements described above as second stage of protection and/or third stage of protection may be integrated into existing HVAC systems.

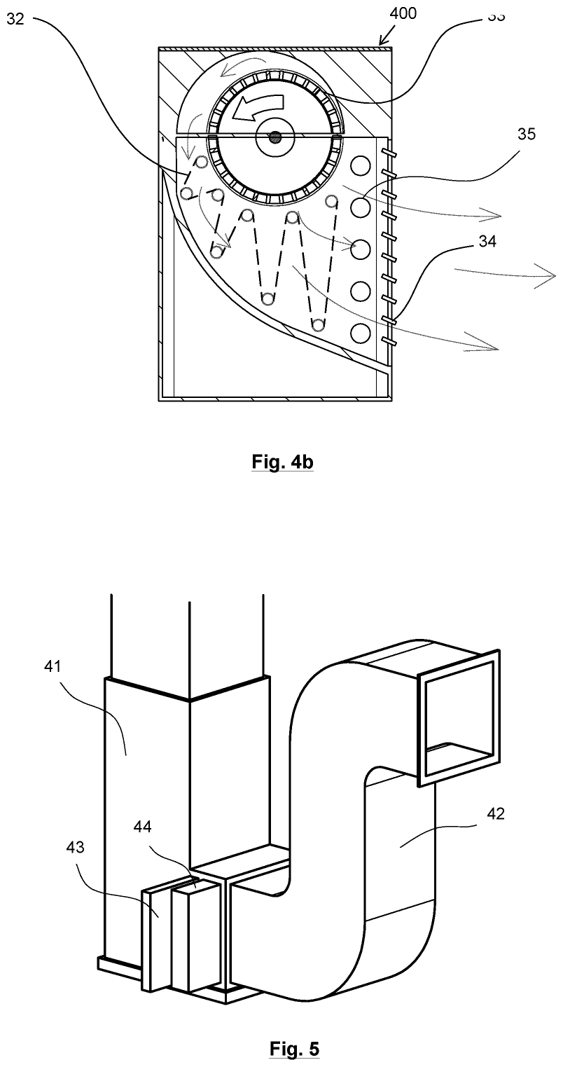

FIG. 5 shows an example of a housing of an HVAC unit 41 having an inlet duct 42, a HEPA filter 44 and an additional specially treated/coated filter 43. An antimicrobial module 43 is also included therein, the module being a specially treated/coated filter 43 configured to contain one or more of the elements described above as providing a second stage or protection (e.g., second filter 3, antimicrobial element 12, set of vortex tubes 22, or some combination thereof). In this embodiment, specially treated/coated filter 43 may be positioned within a pathway of air within an inlet duct 42 of the HVAC unit 41 to provide for eradicating and/or reducing a presence of one or more small pathogens in indoor air.

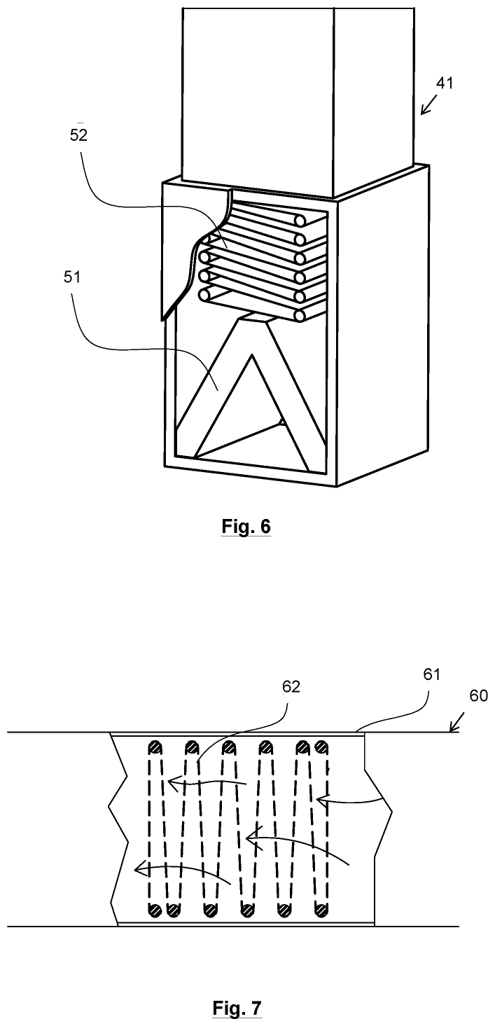

Module 43 that provides for eradicating and/or reducing a presence of one or more small pathogens in indoor air may also be positioned elsewhere in a typical HVAC system, such as may be found in a building (e.g., a residential house). For example, FIG. 6 shows a cutaway view of the HVAC unit 41 including a cooling unit 51 and a module 52 according to another embodiment described herein. Module 52 is similar to module 43 in that it provides a second stage of protection and has an antimicrobial impact on the air. In this example, module 52 is positioned downstream of the cooling unit 51. Again, module 52 may be configured to contain one or more of the elements described above as providing a second stage or protection (e.g., second filter 3, antimicrobial element 12, set of vortex tubes 22, or some combination thereof)

FIG. 7 shows a cross-sectional view of a module 61 according to another embodiment. Here, module 61 is configured to be positioned within a duct 60 of a traditional HVAC system. Module 61 includes extended antimicrobial elements 62 (analogous to antimicrobial elements 12, according to at least one embodiment described herein) retrofitted into an existing HVAC duct system 60. Module 61 is sized and shaped to ensure that air passing through the duct 60 passes through the extended antimicrobial elements 62.

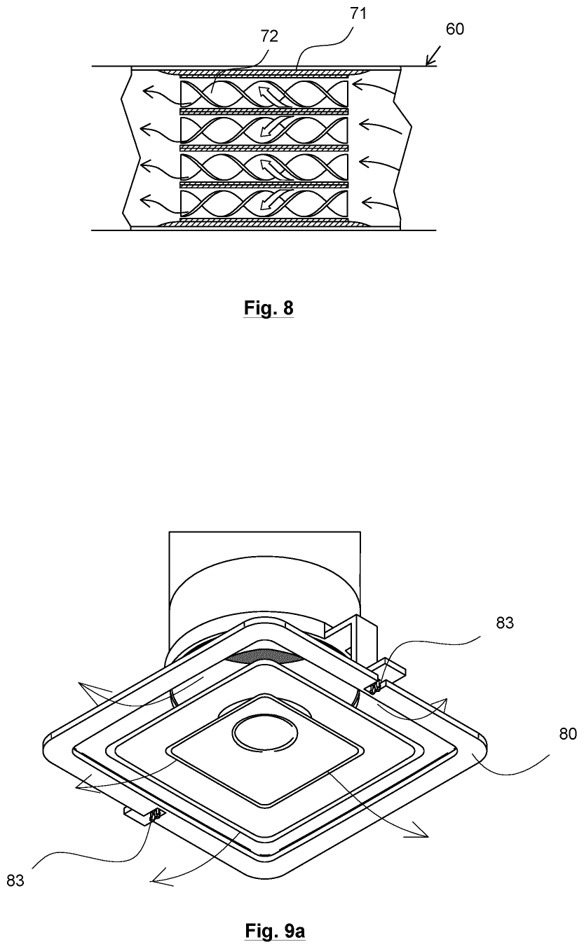

FIG. 8 shows a module 71 according to another embodiment. In this embodiment, module 71 includes vortex tubes 72 (analogous to vortex tubes 22 according to at least one embodiment described herein) retrofitted into an existing HVAC duct 60.

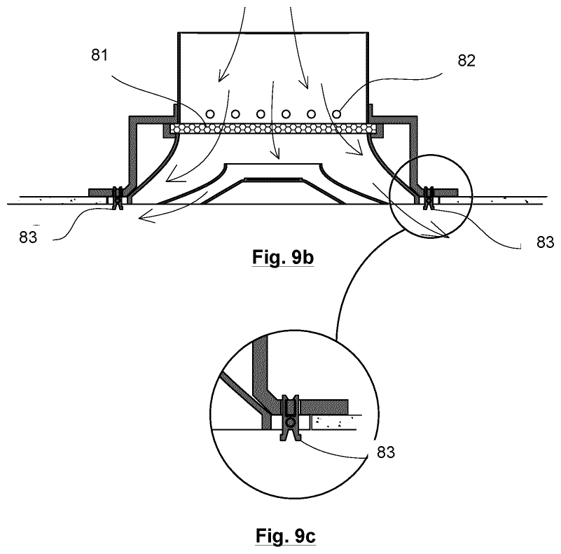

FIGS. 9a and 9b show a ceiling air deflector outlet 80 of an HVAC system. This deflector outlet 80 has clips 83 and can be easily attached/detached with the ceiling duct outlet 84. Herein, elements 81 and 82 are the final stages of air sanitizing control that can be easily accessed for servicing. Elements 81 and 82 may be any element described herein for providing antimicrobial effects to air.

FIG. 9c is a magnified portion of the cross-sectional view of the ceiling air deflector outlet of FIG. 9a.

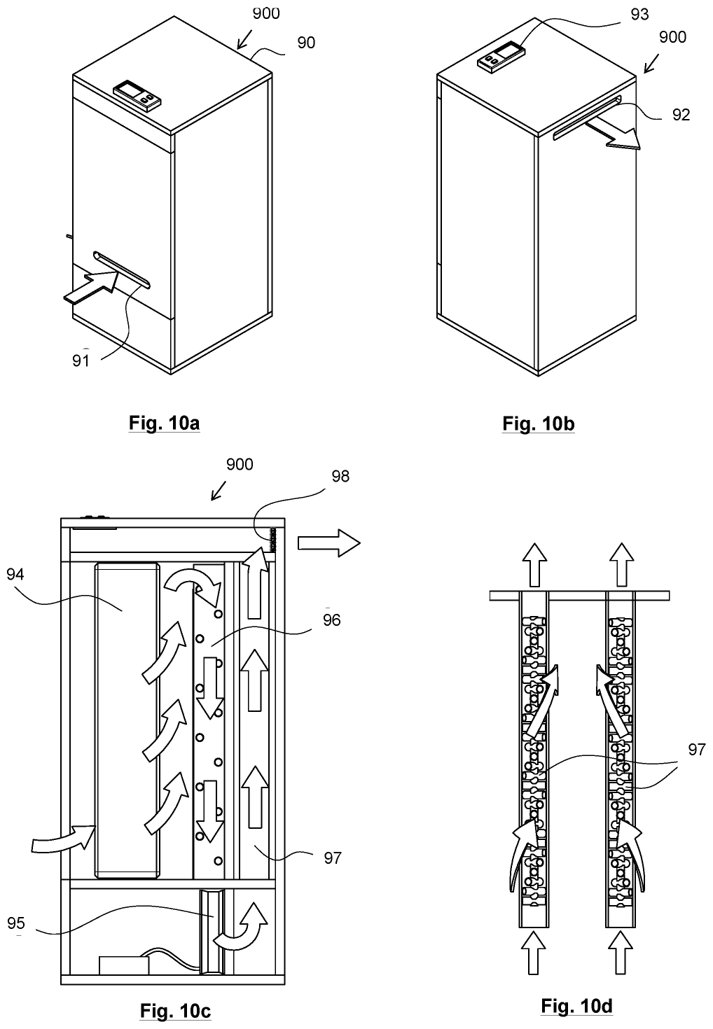

FIG. 10a is a front perspective view of a standalone room air purifying unit 900 having multiple stages of protection, according to at least one embodiment described herein. FIG. 10b is a rear perspective view of the standalone room air purifying unit 900 of FIG. 10a. FIG. 10c is a cross-sectional view of the standalone room air purifying unit 900 of FIG. 10a.

FIG. 10a shows a standalone room air purifying unit 900 with an air inlet slot 91 and an air outlet slot 92 (see FIG. 10b). Standalone room air purifying unit 900 may include a control panel 93 to control operation of fan 95, for example both on and off control and speed control of fan 95.

Air is drawn into housing 90 via inlet 91, and through HEPA filter 94 by fan 95. After being filtered by filter 94, FIG. 10c shows that filtered air travels upwardly through chamber 96 and over an edge 98 that leads to one or more antimicrobial elements positioned within the chamber 96 of housing 90 downstream from the filter 94. Here, the antimicrobial element includes a copper mesh 99. As noted above, copper mesh 99 may be pure copper or may include one or more copper alloys. After travelling downwardly through the copper mesh 99, chamber 96 is configured to redirect the air, after passing through the copper mesh, upwardly to travel through a set of vortex tubes 97 (see FIG. 10d). The set of vortex tubes 97 include left-hand and right-hand spiral vortex tubes, each vortex tube having an inner surface coated with an antimicrobial material, as described above.

After passing upwardly through the set of vortex tubes 97, air is again redirected towards outlet 92. Before passing out of outlet 92, air is passed through a filter 98 positioned adjacent to outlet 92. Filter 98 includes a filter coated with a copper-based material (e.g., copper, or a copper alloy, or the like) for further eradication/reduction of pathogens in the air. Purified air (i.e., air substantially eradicated of pathogens) then exits housing 90 through outlet 92.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

What is claimed is:

1. A standalone unit for providing purified air, the standalone unit comprising:
a housing including an air inlet to receive the air and an air outlet to provide purified air;
a fan positioned within the housing and between the air inlet and the air outlet, the fan configured to control a rate of airflow through the standalone unit;
a filter positioned within the housing and configured for removing particulates from the air received from the air inlet to provide filtered air; and
at least one antimicrobial element positioned within the housing downstream from the filter to receive the filtered air from the filter, the at least one antimicrobial element being configured to receive the filtered air from the filter and eradicate or remove small pathogens from the filtered air to provide the purified air.

2. The standalone unit of claim 1, wherein the antimicrobial element includes a filter having a filter material and an antimicrobial coating at least partially applied to the filter material.

3. The standalone unit of claim 2, wherein the filter material is entirely coated with the antimicrobial coating.

4. The standalone unit of claim 2 or claim 3, wherein the antimicrobial coating comprises one or more salts from an ionic liquid being applied to the filter material.

5. The standalone unit of claim 4, wherein the one or more salts of the antimicrobial coating includes cations and anions crystallized on the filter material.

6. The standalone unit of claim 5, wherein the cations include one or more of imidazolium, pyridnium, piperidinium, pyrrolidinium, quinolinium, morpholinium, quaternary phosphonium and quaternary ammonium cations.

7. The standalone unit of claim 5 or claim 6, wherein the anions includes one or more of tetrafluoroborate, hexafluorophosphate, methylsulfate, octylsulfate, acesulfame, halide ions, bis(trifluoromethyl)sulfonylamide, bis(trifluoromethyl) amide, dicyanamide, and trifluoromethylsulfonate.

8. The standalone unit of any one of claims 3 to 7, wherein the antimicrobial coating includes one or more metal nanoclusters.

9. The standalone unit of claim 9, wherein the metal nanoclusters include copper nanoclusters.

10. The standalone unit of any one of claims 1 to 9, wherein the antimicrobial element includes a copper mesh positioned within the housing downstream from the filter.

11. The standalone unit of any one of claims 1 to 10, wherein the antimicrobial element includes a set of vortex tubes.

12. The standalone unit of claim 11, wherein the set of vortex tubes each have an inner surface at least partially coated with an antimicrobial material.

13. The standalone unit of claim 12, wherein the set of vortex tubes each have an inner surface entirely coated with the antimicrobial material.

14. The standalone unit of claim 13, wherein the antimicrobial material a copper-based material.

15. The standalone unit of claim 14, wherein the antimicrobial material is copper.

16. The standalone unit of any one of claims 11 to 15, wherein the set of vortex tubes include left-hand and right-hand spiraling vortex tubes.

17. The standalone unit of any one of claims 11 to 16 further comprising a third filter positioned downstream from the set of vortex tubes and adjacent to the outlet, the third filter having a copper-based material.

18. A module for a heating, ventilation and air conditioning (HVAC) system for purifying air of the HVAC system, the module being configured to be positioned within a portion of the HVAC system to receive the air of the HVAC system, the module comprising:
an antimicrobial element configured to eradicate or remove small pathogens from the air to provide purified air.

19. The module of claim 18, wherein the antimicrobial element includes a filter having a filter material and an antimicrobial coating at least partially applied to the filter material.

20. The module of claim 19, wherein the filter material is entirely coated with the antimicrobial coating.

21. The module of claim 18 or claim 19, wherein the antimicrobial coating comprises one or more salts from an ionic liquid being applied to the filter material.

22. The module of claim 21, wherein the one or more salts of the antimicrobial coating includes cations and anions crystallized on the filter material.

23. The module of claim 22, wherein the cations include one or more of imidazolium, pyridnium, piperidinium, pyrrolidinium, quinolinium, morpholinium, quaternary phosphonium and quaternary ammonium cations.

24. The module of claim 22 or claim 23, wherein the anions includes one or more of tetrafluoroborate, hexafluorophosphate, methylsulfate, octylsulfate, acesulfame, halide ions, bis(trifluoromethyl)sulfonylamide, bis(trifluoromethyl) amide, dicyanamide, and trifluoromethylsulfonate.

25. The module of any one of claims 20 to 24, wherein the antimicrobial coating includes one or more metal nanoclusters.

26. The module of claim 25, wherein the metal nanoclusters include copper nanoclusters.

27. The module of any one of claims 18 to 26, wherein the antimicrobial element includes a copper mesh positioned within the housing downstream from the filter.

28. The module of any one of claims 18 to 26, wherein the antimicrobial element includes a set of vortex tubes.

29. The module of claim 28, wherein set of vortex tubes each have an inner surface at least partially coated with an antimicrobial material.

30. The module of claim 29, wherein the set of vortex tubes each have an inner surface entirely coated with the antimicrobial material.

31. The module of claim 30, wherein the antimicrobial material a copper-based material.

32. The module of claim 31, wherein the antimicrobial material is copper.

33. The module of any one of claims 28 to 32, wherein the set of vortex tubes include left-hand and right-hand spiraling vortex tubes.

34. The module of any one of claims 18 to 33, wherein the module is configured to be positioned within a duct of the HVAC system.

35. The module of any one of claims 18 to 33, wherein the module is configured to be positioned within a ceiling air deflector of the HVAC system.
